(19) United States
(12) Patent Application Publication
(10) Pub. No.: US 2017/0319235 A1
(43) Pub. Date: Nov. 9, 2017

(54) BLADDER TREATMENT METHODS AND DEVICES

(71) Applicant: Amphora Medical, Inc., Maple Grove, MN (US)

(72) Inventors: Eric S. Whitbrook, St. Paul, MN (US); John C. Knudson, Minnetonka, MN (US); Edwin J. Hlavka, Minneapolis, MN (US); Thomas V. Ressemann, Edina, MN (US)

(73) Assignee: Amphora Medical, Inc., Maple Grove, MN (US)

(21) Appl. No.: 15/589,793

(22) Filed: May 8, 2017

Related U.S. Application Data
(60) Provisional application No. 62/448,790, filed on Jan. 20, 2017.
(60) Provisional application No. 62/332,820, filed on May 6, 2016.

(51) Int. Cl. A61B 17/42; A61B 18/18; A61B 17/00; A61N 7/02; A61B 18/14; A61M 31/00; A61B 18/00

(52) U.S. Cl. CPC A61B 17/42; A61B 18/1485; A61B 18/1815; A61B 17/00234; A61N 7/022; A61M 31/002; A61B 2017/00278; A61B 2018/00434; A61B 2018/00523; A61B 2018/1861; A61M 2210/1078; A61M 2210/1475; A61B 2018/00577

(57) ABSTRACT

Devices and methods that provide minimally-invasive approaches to performing treatments on soft tissue, such as that found in the bladder using various natural access paths such as transvaginal and transurethral. Treatments include the application of energy to nerves found in, under and around the trigone region of the bladder.

### RELATED APPLICATIONS

This application claims benefit of and priority to U.S. Provisional Application Ser. No. 62/332,820 filed May 6, 2016 entitled Bladder Treatment Methods and Devices, and to U.S. Provisional Application Ser. No. 62/448,790 filed Jan. 20, 2017 entitled Bladder Treatment Methods and Devices both of which are hereby incorporated by reference herein in their entireties.

### BACKGROUND OF THE INVENTION

Certain abnormalities associated with the bladder, such as overactive bladder (OAB) are believed, in part, due to pathologies associated with nerves that communicate with the bladder. In particular, afferent nerves that innervate the bladder wall. For example, significant innervation exists in the trigone area of the bladder.

It is believed that if the innervation associated with the trigone or other regions of the bladder can be interrupted, certain bladder abnormalities may be ameliorated.

In addition to treatments that interrupt the nerve endings directly within the bladder wall, treatments that interrupt the larger nerve branches associated with regions of the bladder such as the trigone may also improve symptoms associated with overactive bladder.

Many of the nerve branches that are in communication with the trigone region of the bladder are believed to course from the trigone region deeply and laterally toward the lateral interface of the vaginal and bladder walls, and from there superiorly towards the ureteral orifices and then along the ureters.

The nerves associated with the trigone region of the bladder may be approached favorably from within the vagina, in addition to approaching from the bladder or other anatomical locations. Approaches from the vagina may target the nerves directly as they course from the trigone region, or they may target the nerves as they course more deeply and more laterally from the trigone region.

### OBJECTS AND SUMMARY OF THE INVENTION

Methods and devices for interrupting the nerves and nerve branches associated with regions of the bladder such as the trigone are contemplated. Interruption may be achieved using energy delivery to locally ablate or interrupt the functioning of the nerves and nerve tissues.

One embodiment of the invention provides a method of treating a bladder condition that includes placing a treatment device in a patient's vagina such that a front face of the device faces the patient's bladder and delivering a treatment modality from regions of the device that are lateral to the front face of the treatment device such that said treatment modality affects biological functionality of nerve tissue lateral of the vagina.

Delivering a treatment modality from the treatment device may involve placing at least one energy delivery element from the treatment device into a lateral wall of the vagina; and, delivering energy to tissue within a desired proximity of the delivery element.

An aspect of this method may involve delivering a pharmaceutical agent to a lateral wall of the vagina.

Alternatively, or additionally, RF energy, and/or microwave energy, and/or ultrasonic energy may be delivered to the lateral wall from the delivery element.

It is also an aspect of the invention to deliver the treatment modality from the front face of the treatment device.

Another aspect of the invention is a method of treating a bladder condition that includes placing a treatment device in a ureter and treating nerve tissue in a region away from the epithelium of the ureter while protecting the epithelium from treatment. The step of treating nerve tissue in a region outside of the ureter may involve placing at least one energy delivery element in a lateral wall of the ureter and delivering energy to the lateral wall from the delivery element.

Treating nerve tissue in a region outside of the ureter may include delivering a pharmaceutical agent to a lateral wall of the ureter.

Delivering energy to the lateral wall may include delivering RF energy to the lateral wall from the delivery element, and/or delivering microwave energy to the lateral wall from the delivery element and/or delivering ultrasonic energy to the lateral wall from the delivery element.

One aspect of this method includes placing an expandable balloon in a ureter that includes at least on energy delivery element.

Another aspect of this method includes cooling a surface of said ureter. Yet another aspect of the invention provides a method of treating overactive bladder (OAB) in a female patient by placing a treatment device having at least one surface electrode in the vagina to a position where the at least one electrode is faces the trigone region of the bladder, the device having an internal lumen configured for receiving a cooling fluid; activating the at least one electrode to affect nerve functionality; and infusing cooling fluid into the internal lumen. The step of activating the at least one electrode may occur prior to, after; or simultaneously with the step of infusing cooling fluid into the internal lumen. The step of activating the at least one electrode may include activating at least two surface electrodes that operate in a bipolar RF mode

Another aspect of the invention includes a method of treating overactive bladder (OAB) in a female patient by placing a visualization device in the vagina to a position adjacent the bladder; identifying the location of a ureter and noting the position of the ureter relative to the patient's anatomy or the position of the visualization device; deploying at least one energy delivery element to a position relative to the ureter; and activating the energy delivery element to treat nerve tissue associated with OAB.

The energy delivery element may include an RF electrode that is deployed into the vaginal wall to a position adjacent the trigone region of the bladder.

The energy delivery element may include a surface RF electrode that is positioned against the vaginal wall to a position adjacent the trigone region of the bladder.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a cutaway view of a female bladder showing the trigone region;

FIG. 2 is a cutaway view of a female bladder showing the trigone region and a vagina underlay in phantom lines;

FIG. 3 is a diagram of anatomical features apparent in a histology cross section along section line B-B of FIG. 2;

FIG. 4 is a diagram of anatomical features apparent in a histology cross section along section line C-C of FIG. 2;

FIG. 5 is a cutaway view of the female bladder showing the trigone region, a vagina underlay and areas of high nerve concentration;

FIG. 6 is a cutaway view of the female vagina showing the trigone area as an overlay and highlighting areas of high nerve concentration;

FIG. 7 is an elevation of an embodiment of a device of the invention;

FIG. 8 is an elevation of an embodiment of a device of the invention;

FIG. 9 is an elevation of an embodiment of an energy element of the invention;

FIG. 10 is an elevation of an embodiment of an energy element of the invention;

FIG. 11 is an elevation of an embodiment of an energy element of the invention;

FIG. 12 is a perspective view of an embodiment of a device of the invention;

FIG. 13 is a perspective view of an embodiment of a device of the invention;

FIG. 14 is an end view of an embodiment of a device of the invention;

FIG. 15 is an end view of an embodiment of a device of the invention;

FIG. 16 is an elevation of an embodiment of a device of the invention;

FIG. 17 is an elevation of an embodiment of a device of the invention;

FIG. 18 is an elevation of an embodiment of a device of the invention;

FIG. 19 is an elevation of an embodiment of a device of the invention;

FIG. 20 is an elevation of an embodiment of a device of the invention;

FIG. 21 is an elevation of an embodiment of a device of the invention;

FIG. 22 is an elevation of an embodiment of a guide/handle of the invention;

FIG. 23 is an end view of an embodiment of a guide/handle of the invention;

FIG. 24 is a end view of an embodiment of a guide/handle of the invention being placed into a patient;

FIG. 25 is an elevation of an embodiment of a device of the invention;

FIG. 26 is an elevation of an embodiment of a device of the invention;

FIG. 27 is an elevation of an embodiment of a device of the invention;

FIG. 28 illustrates a histological cross-section of the bladder and vagina showing the sub-trigone region;

FIG. 29 illustrates a side cross-section of the bladder and vagina identifying the sub-trigone region;

FIG. 30 illustrates a front cross-section of the bladder identifying the sub-trigone region;

FIG. 31 is an elevation of an embodiment of a device of the invention;

FIG. 32 is an elevation of an embodiment of a device of the invention;

FIG. 33 is an elevation of an embodiment of a device of the invention;

FIG. 34 is an elevation of an embodiment of a device of the invention;

FIG. 35 is an elevation of an embodiment of a device of the invention;

FIG. 36 is an end view of the embodiment of FIG. 35;

FIG. 37 is an elevation of an embodiment of a device of the invention;

FIG. 38 is a depiction of an image taken with the device of FIG. 37;

FIG. 39 is an elevation of an embodiment of a device of the invention;

FIG. 40 is a depiction of an image taken with the device of FIG. 39;

FIG. 41 is an elevation of an embodiment of a device of the invention;

FIG. 42 is an elevation of an embodiment of a device of the invention;

FIG. 43 is an elevation of an embodiment of a device of the invention;

FIG. 44 is an elevation of an embodiment of a device of the invention;

FIG. 45 is an end view of the embodiment of FIG. 44;

FIG. 46 is an elevation of an embodiment of a device of the invention emitting energy in a ureter; and,

FIG. 47 is an elevation of an embodiment of a device of the invention emitting energy in a ureter.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

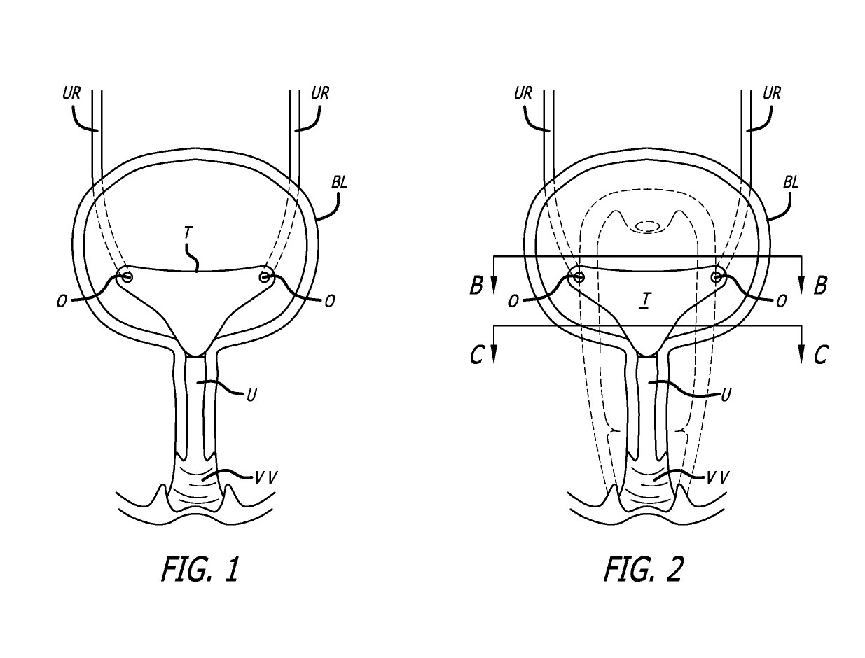

FIG. 1 is a partial section view showing a female bladder BL, urethra U, bladder wall W trigone region T, ureteral orifices O, and ureters UR, as well as the lower portion of the vaginal vault VV.

FIG. 2 illustrates the bladder anatomy of FIG. 1, superimposed on the vaginal wall VW (dotted line), which is below the urethra and trigone region. Section lines B-B and C-C are described below.

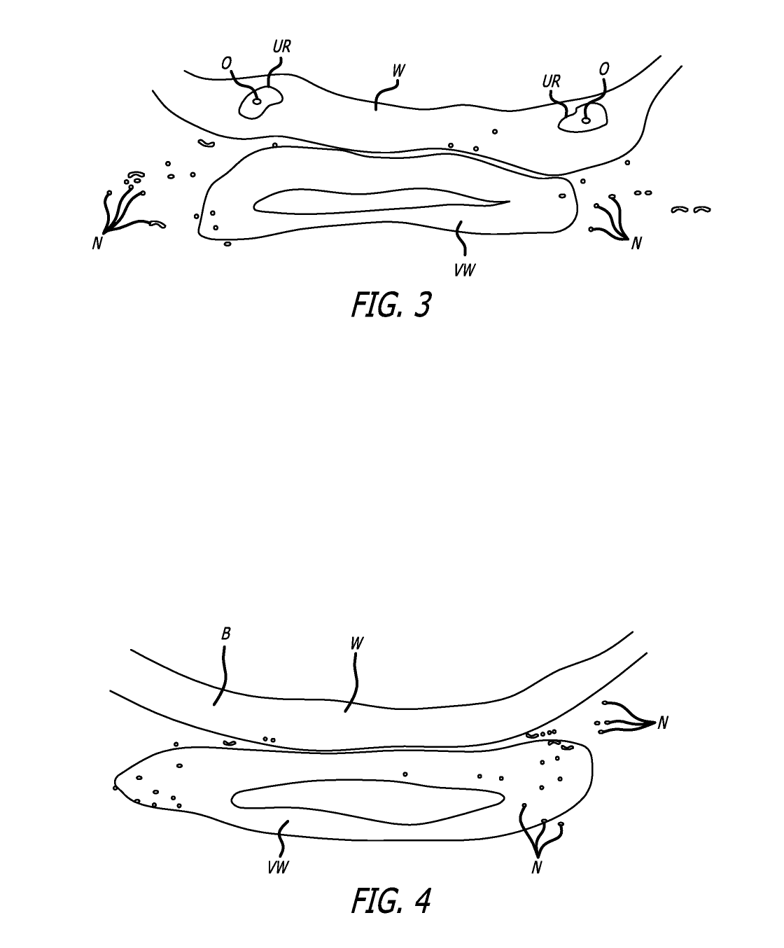

FIG. 3 is a diagram of anatomical features that appear in a histology cross section along section line B-B of FIG. 2. This location is somewhat proximal (superior and posterior) of the trigone region of the bladder wall. The vaginal wall VW and inferior bladder wall W can be seen. Note that the histology section is in two parts due to limitations in preparing of the histology sections and has some cutting artifacts. The ureters UR can be seen, embedded within the bladder wall VV. A staining technique which stains nerve cells was performed, and nerves and ganglia features N greater than about 50 microns were marked. Note that many of these nerves are lateral of the vagina, as well as lateral and oblique towards the bladder walls, and some may be within the vaginal wall VW. These nerves are associated with the nerve endings associated with the trigone region of the bladder. Interruption of the nerves at any point from the nerve endings themselves, to the larger trunks as shown in this figure may alter bladder function and provide relief from bladder conditions such as overactive bladder.

The anatomical features highlighted and approximated, including the bladder wall W, the vaginal wall VW, and exemplary small nerves and ganglia N. The dashed line represents the areas A with fairly high concentrations of nerves believed associated with bladder function. The dashed lines are general areas A which may be a “lateral” region of interest for modifying as a means of treating a bladder condition.

FIG. 4 is a diagram of anatomical features that appear in a histology cross section along section line C-C of FIG. 2. This location includes a portion of the trigone region T of the bladder B, distal of the ureteral orifices. Therefore the ureters are not visible in this section. Note that the walls W of the bladder B and vaginal wall VW are clearly visible, as well as numerous small nerves and ganglia N laterally disposed of the vaginal wall VW.

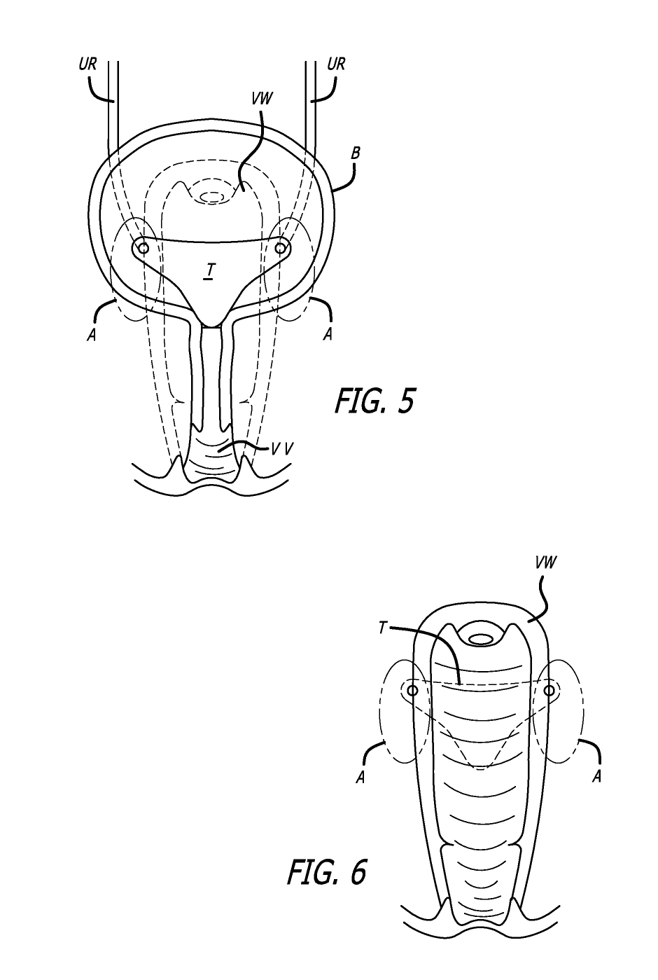

FIG. 5 illustrates the location A of the small nerves and ganglia in relation to the top view of the bladder B and the vaginal wall VW. Treatments that target nerve structures in this “lateral” region(s) of interest may benefit bladder conditions such as overactive bladder.

FIG. 6 shows the “lateral” region(s) of interest A of FIG. 5 with just the vaginal wall VW shown. The trigone region T of the bladder (superior to the vagina) is shown in phantom (small dashed line).

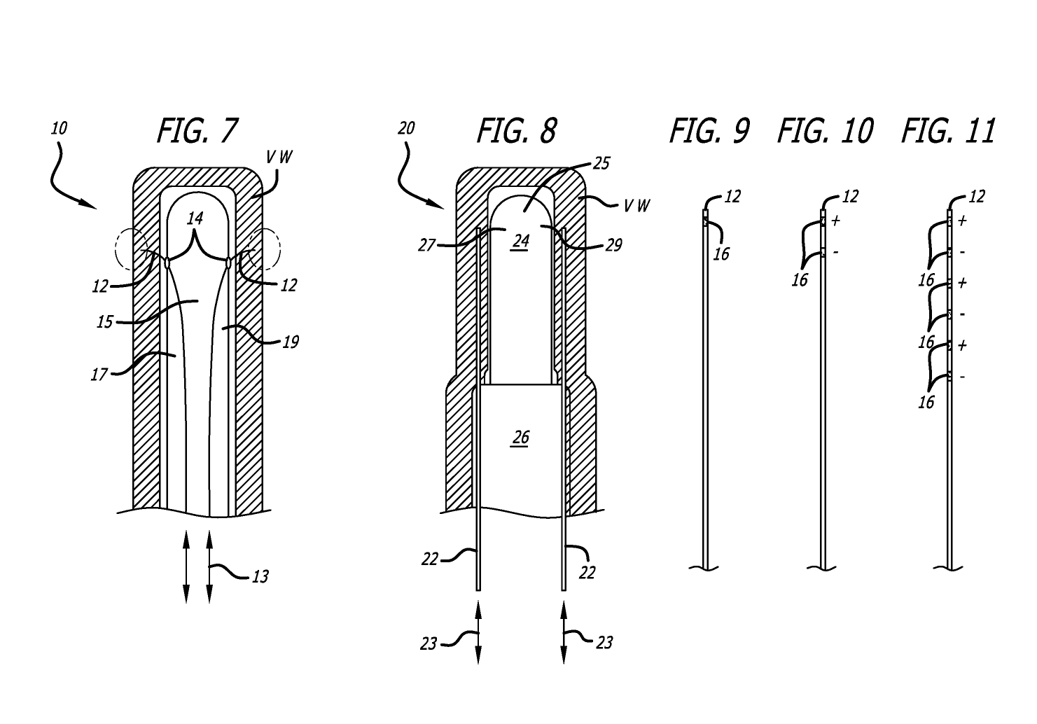

A treatment device 10 configured to be placed in the vaginal wall VW is illustrated in FIG. 7. Treatment device 10 may include one or more energy delivery elements 12 for placement within or through the wall of the vagina VW, to deliver energy within and/or external of the vaginal wall. For example, energy delivery element(s) 12 may be positioned in the lateral sides of the vaginal wall, as shown. Such energy may alter nerves or nerve tissue within a “lateral” region of interest. As used herein, “lateral” is used to refer to the area of the vagina that is adjacent to that area of the vagina closest to the bladder. For purposes of clarity, the portion of the vagina that faces the bladder will be referred to as the “front” or “superior” face of the vagina, while the areas adjacent to the front will be referred to as the “lateral” areas. If one were to assume the vagina was roughly a circular tube, and assign the front face of the vagina as the 12 o'clock position, the term “lateral” would refer to areas counterclockwise of 12 o'clock, for example from approximately 11 o'clock to approximately 9 o'clock, and clockwise of the 12 o'clock position, for example from approximately 1 o'clock to approximately 3 o'clock. In FIG. 11, reference numeral 15 is assigned to the front face of the device 10, which is positioned to correspond to the front or superior face of the vagina, or the 12 o'clock position. Numerals 17 and 19 are thus lateral areas, also referred to as “lateral faces” of the device. One skilled in the art will understand that the device may have a cross-section that is circular, oval, elliptical or the like, without clear “faces” distinguished by edges, and that as used herein, “faces” are being used merely to distinguish areas of the various embodiments that are at the 12 o'clock position, from areas immediately adjacent to the 12 o'clock position, without implying that an edge separates the various faces.

Energy delivery elements may be configured to deliver radio frequency (RF) energy, resulting in localized heating of tissue. Energy delivery element(s) 12 may be configured to be advanceable through apertures 14 in the device 10 to an extended state (shown) from a retracted state (not shown), as indicated by arrows 13. After energy delivery, energy delivery element(s) 12 may be retracted into treatment device 10, and removed from the vagina. Such a treatment may interrupt nerves associated with overactive bladder. The energy delivery elements may be positioned within the vaginal wall VW, on the outer aspect of the vaginal wall, or external of the vaginal wall. Even if the energy delivery elements are within the vaginal wall as illustrated, the effect of the energy delivery may spread to the extra-vaginal tissue to modify nerves within that region of interest.

Energy delivery element(s) 12 may also be configured to deliver other types of energy, such as microwave, ultrasonic, cryogenic, or other energy forms that may suitably interrupt the nerve tissues in a region of interest.

Energy delivery element(s) 12 may alternately be conduits for delivery of pharmacologic agents capable of disrupting nerve tissues, such as anesthetics or toxins (e.g. Botox), either together with energy, or instead of energy.

If the energy delivery element(s) 12 are configured for delivery of RF energy, they may each include a single contact 16 to form a monopolar electrode (FIG. 9), or a pair of contacts 16 to form a bipolar electrode (FIG. 10). Alternately, the energy delivery element(s) may include several contacts 16 of alternating polarity (FIG. 11), which is capable of heating a relatively long region of tissue. Also, multiple monopolar needles may be paired in such a way that they deliver bipolar energy between them.

FIG. 8 illustrates an embodiment of a treatment device 20 configured for placement in the vaginal wall VW. One or more energy delivery elements 22 may be extended longitudinally into the vaginal wall VW into a region of interest, for example within or external to the lateral sides of the vaginal wall VW. Treatment device may include a reduced diameter portion 24 distally, with a front face 25 and lateral faces 27 and 29, and may further include suction means (not shown) to bring tissue radially inwards. Energy delivery element(s) 22 may extend, as indicated by arrows 23, from a proximal portion 26 of treatment device and penetrate tissue longitudinally within the tissue. Energy delivery element(s) 22 may also be configured to extend at an angle relative to the body of the treatment device, and may also extend to tissue outside of the vaginal wall. Energy delivery element(s) may be configured to deliver various forms of energy, for example RF energy. As such, the energy delivery element(s) 22 may be monopolar, bipolar, or a multiple bipolar arrangement, as shown in FIGS. 9, 10, and 11. Furthermore, energy delivery element(s) 22 may be positioned at multiple positions to treat larger zones. If energy delivery elements are disposed within the vaginal wall, it is contemplated that the energy delivered may extend to tissue outside of the vaginal wall.

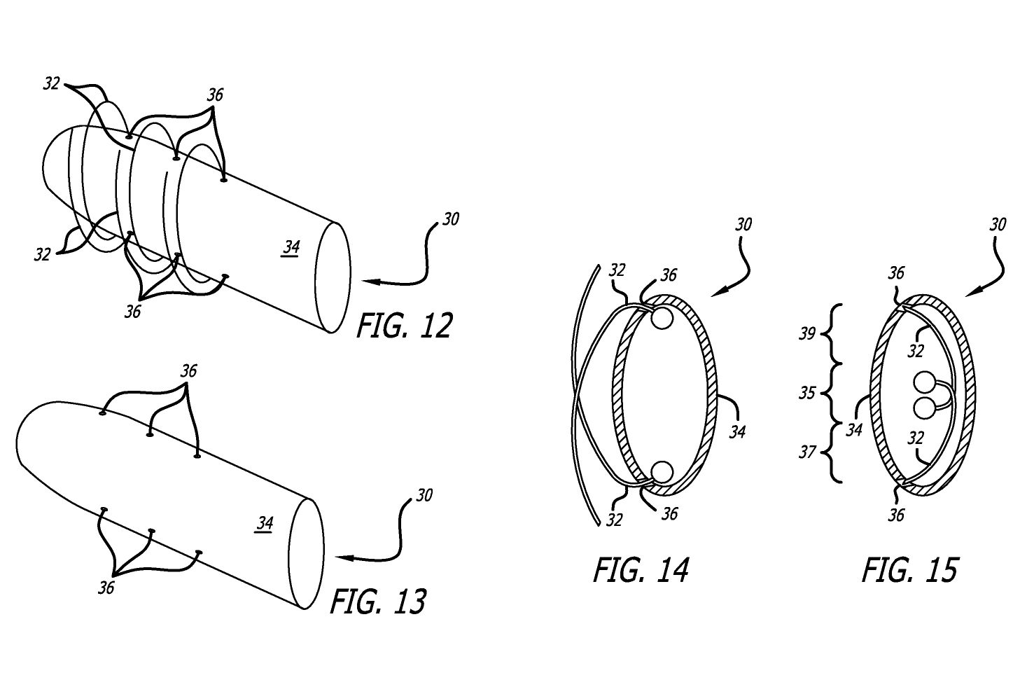

FIG. 12 illustrates another embodiment 30 of a treatment device configured for placement in the vagina. One or more energy delivery elements 32 may be extended from the body 34 through apertures 36. Energy delivery element(s) 32 may extend in a generally circumferential direction and penetrate the vaginal wall, or extend into tissue outside of the vaginal wall. FIG. 13 illustrates the body of the treatment device 30 with the energy delivery element(s) in a retracted position, which would be suitable for initial entry into the vagina, and after the energy delivery treatment is performed, prior to removal. The embodiment 30 of FIG. 12 may impact nerves that are both laterally disposed of the vagina, as well as nerves that extend more medially in the interface of the vagina and bladder.

FIGS. 14 and 15 show the treatment device 30 with energy delivery element(s) 32 in retracted (FIG. 14) and extended (FIG. 15) positions. Energy delivery element(s) 32 may be connected to deployment arm(s), which may be manipulated from a proximal end of the treatment device (not shown). As with other treatment device embodiments, energy delivery element(s) 32 may be configured to deploy numerous types of energy, such as RF energy, and as such may be have a monopolar or bipolar configuration. FIG. 15 shows the front face 35 and the lateral faces 37 and 39 of the body 34 of the device.

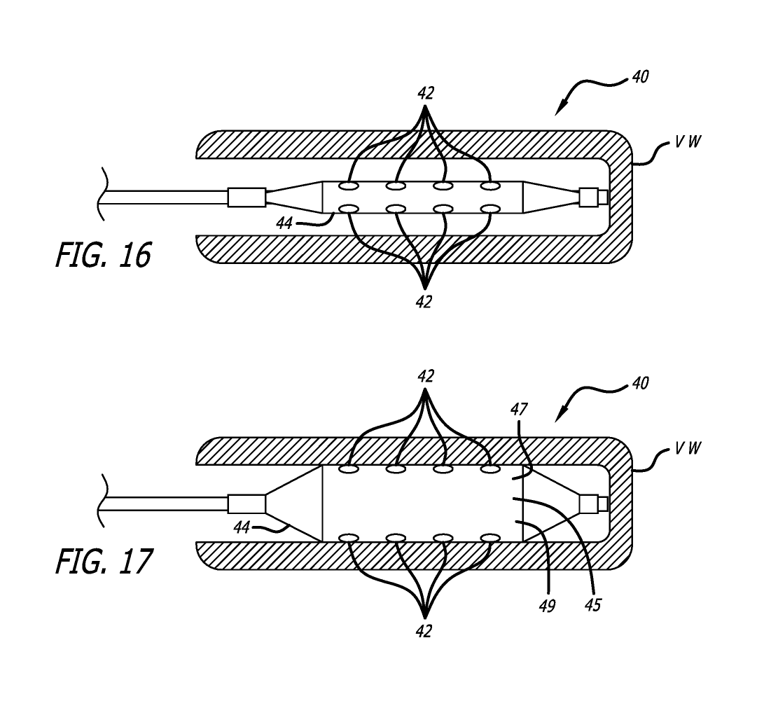

Another embodiment 40 of a treatment device configured for placement in the vaginal wall VW is shown in FIGS. 16 and 17. Treatment device 40 may include an expandable balloon 44 with one or more energy delivery elements 42 connected to the surface. Energy delivery element(s) 42 may be located to treat a region of interest within or external to the vagina wall, for example, along the lateral sides of the vagina. Embodiment 40 includes a front face 45 and lateral faces 47 and 49. Treatment device 40 may be positioned in the vagina in a deflated condition, as shown in FIG. 16. Once positioned, the balloon 44 may be expanded, placing the energy delivery elements 42 in contact with the wall. Energy delivery element(s) 42 may then be activated, delivering energy to and through the tissue. Optionally, the balloon 44 may be kept cool with suitable cooling fluid so as to protect most or all of the surface tissue from being energized. Energy delivery element(s) 42 may be configured to deliver various forms of energy, such as RF energy. As such, energy delivery element(s) 42 may be configured to deliver monopolar or bipolar RF energy.

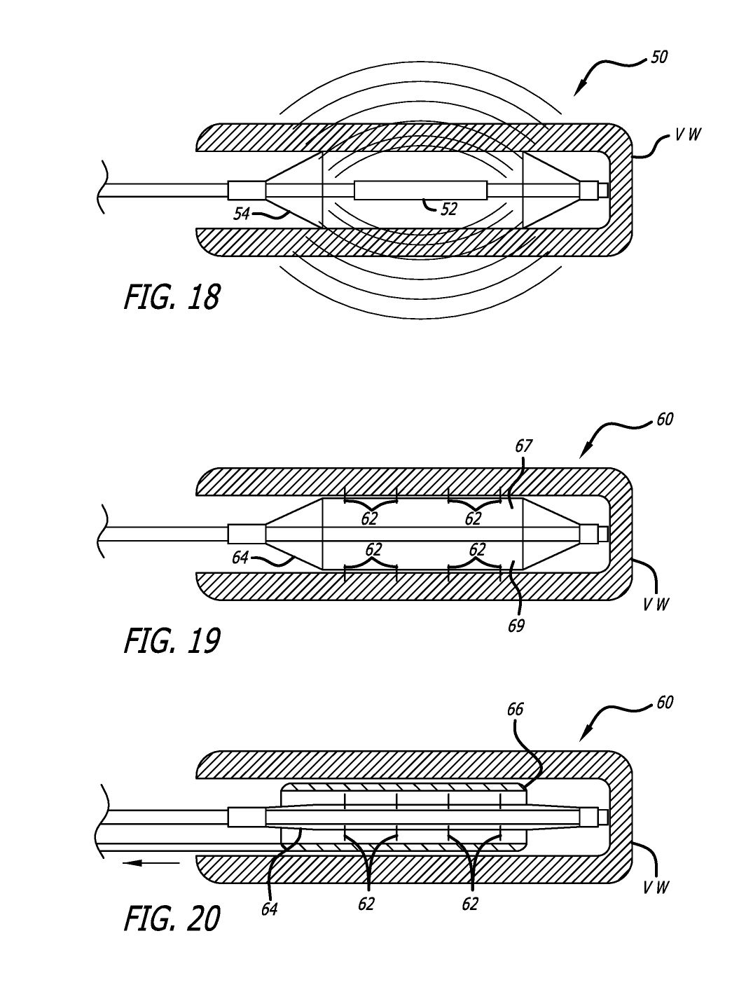

FIG. 18 illustrates an embodiment of an expandable treatment device 50 incorporating an alternative energy delivery element 52. The energy delivery element 52 may be located centrally, as opposed to on the surface of the expandable component 54. Locating the energy delivery element 52 centrally within the expandable component 54, controls the separation between the element 52 and the vaginal walls VW. Here, energy delivery element 52 may be a microwave emitter. Microwave energy emits radially outward to treat (heat) some or all of the vaginal wall, and may extend to tissue outside of the vaginal wall. Optionally, cooling fluid within the expandable component 54 may keep the surface or all of the vagina cool, so as to minimize any thermal damage on the surface or within the vagina, while the energy treats tissue outside of the vaginal wall. The energy delivery element of the treatment device shown in FIG. 18 may also be configured to deliver ultrasound energy. Similar to microwave energy, the energy would radiate outward to treat some or all of the vaginal wall or tissue outside of the vaginal wall. The expandable component 54 may be an inflatable balloon, a mechanically expandable device, or a self-expanding device. Depending on the type of delivery element 52 being used, it is envisioned that the device 52 be alternatively located on the surface of the expandable component 54 and my also comprise a plurality of elements 52.

FIG. 19 shows yet another embodiment 60 of a treatment device configured for placement within a vagina. Treatment device 60 may include mechanically expandable or inflatable balloon 64, with one or more penetrating energy delivery elements 62 secured to the surface. Expansion of the balloon 64 urges the energy delivery element(s) 62 on the lateral faces 67 and 69 of the device to penetrate the tissue to a region of interest for treatment, for example the lateral sides of the vaginal wall VW. The energy delivery elements 62 may be configured for delivery of RF energy. Electrical contacts (not shown) near the tips of the energy delivery elements 62 may heat tissue within the vaginal wall VW, away from the surface, to treat tissue within and/or outside of the vagina to interfere with nerve tissue in those regions. Optionally, a protective sheath 66 may be incorporated for delivery into and from the vaginal wall VW, as seen in FIG. 20.

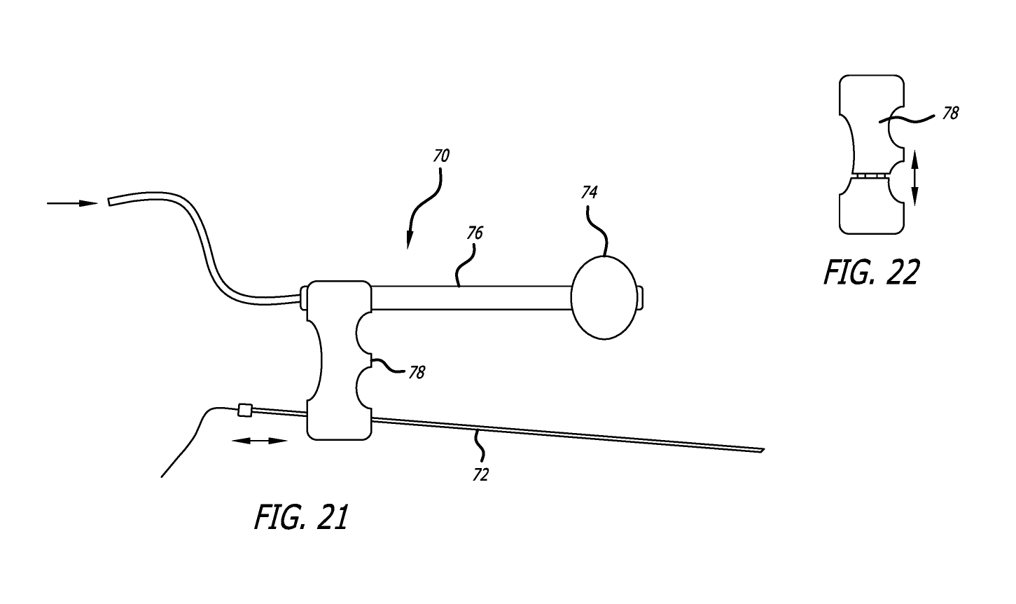

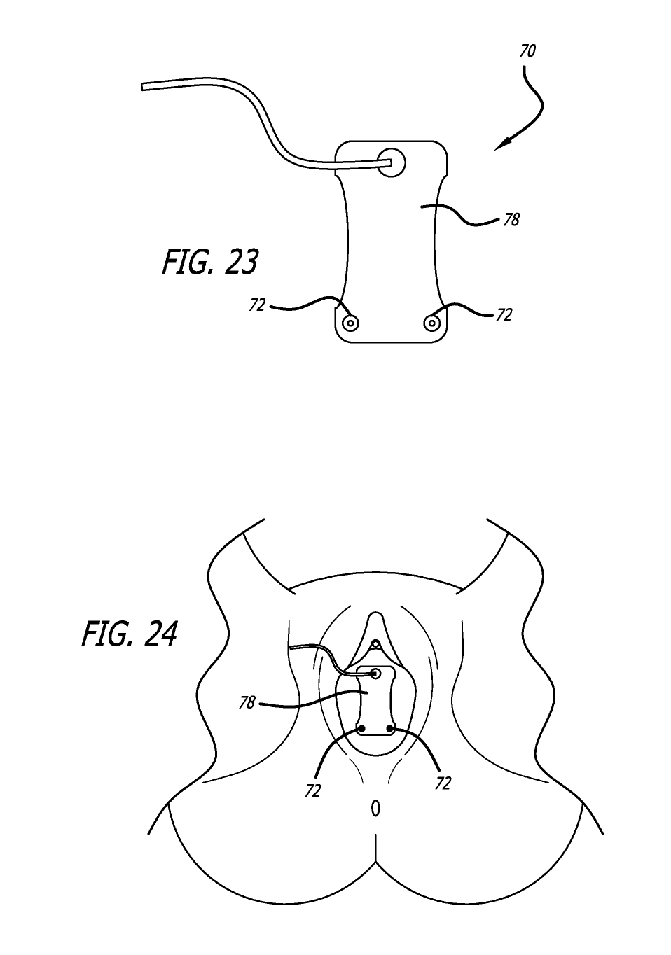

FIG. 21 illustrates a treatment device 70 configured to treat a region of interest by delivery of energy to nerve tissue associated with various bladder conditions such as overactive bladder. Treatment device 70 is configured to place one or more energy delivery elements 72 into vaginal wall tissue, for example into the lateral walls of the vagina at a position near the bladder trigone. Energy delivery elements 72 may be delivered along a trans-perineal route laterally of the vaginal surface. For example, this trans-perineal route could involve penetrating the perineum, avoiding the vaginal entroitus. Alternatively, energy delivery elements 72 may be configured to penetrate the interior of the vaginal wall at some location inside the vagina, and then extend into vaginal tissue or to tissue outside of the vagina, ending in a region of interest for treatment. Treatment device 70 may include a urethral shaft 76, an inflatable positioning balloon 74, a guide/handle 78, and one or more advancable energy delivery elements 72. FIG. 23 shows treatment device from the back, and the lateral arrangement of the energy delivery elements 72. In use, the energy delivery elements 72 may be initially retracted, the inflatable positioning balloon 74 is placed in the bladder, inflated and positioned adjacent the bladder neck. The guide/handle 78 is oriented posteriorly toward the vaginal opening, thus precisely placing the energy delivery elements in a proper orientation for trans-perineal delivery, as best seen in FIG. 24. Energy delivery elements 72 are then advanced, placing them within the vaginal wall or into tissue outside of the vagina at a location of interest. Even if the energy delivery elements reside in vaginal tissue, the energy may radiate outward to alter nerve tissue outside of the vaginal wall.

Energy delivery elements 72 may be configured to deliver various forms of energy, such as RF energy. For delivery of RF energy, elements may be configured for monopolar or bipolar RF. Energy delivery elements may be repositioned in multiple locations to create multiple local treatment zones. Elements may also include multiple electrodes to treat a larger/longer zone. Also, as with other embodiments of treatment devices described here, energy delivery elements may be configured to deliver a pharmacologic agent in addition to, or instead of energy. For example, elements could be configured for delivery of an anesthetic or a toxin such as Botox.

The guide/handle 78 of the treatment device 70 of FIG. 21 serves to precisely position the energy delivery elements relative to a stable anatomic structure, such as the bladder neck and urethra. Proper alignment of the energy delivery elements may need to be adjusted and set differently for different patients. Guide/handle 78 may include one or more adjustments, as shown in FIG. 22. Here, the vertical height may be adjusted by elongating the guide/handle 78. Although not shown, guide/handle 78 may further be configured to adjust the entry angle of the energy delivery elements relative to the shaft. Also, the lateral spacing of the energy delivery elements may be facilitated by an adjusting mechanism.

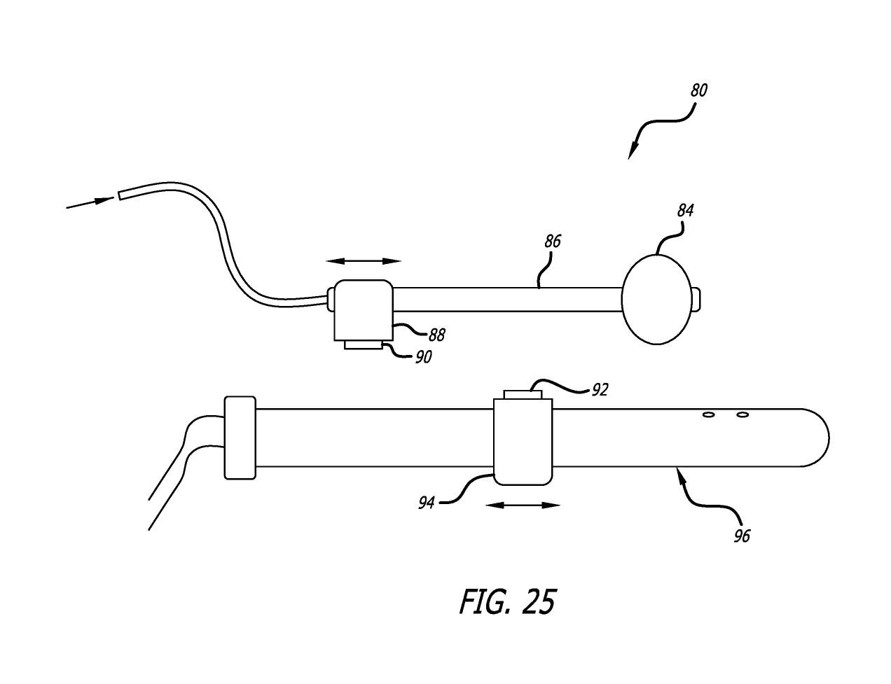

Aspects of the precise positioning provided by the embodiment 70 of FIG. 21 can also be adapted to the trans-vaginal treatment devices described above. An example embodiment 80 is shown in FIG. 25. Inflatable positioning balloon 84 is on a shaft 86 configured for trans-urethral placement at the bladder neck. A urethral guide/handle 88 is adjustably securable to the shaft, and includes a mating portion 90, to be mated with a corresponding mating portion 92 of a vaginal guide/handle 94 that is adjustably securable to a vaginal treatment device 96. Note that the vaginal treatment device 96 shown is similar to that shown in FIG. 7, but could be similar to the other trans-vaginal treatment devices also.

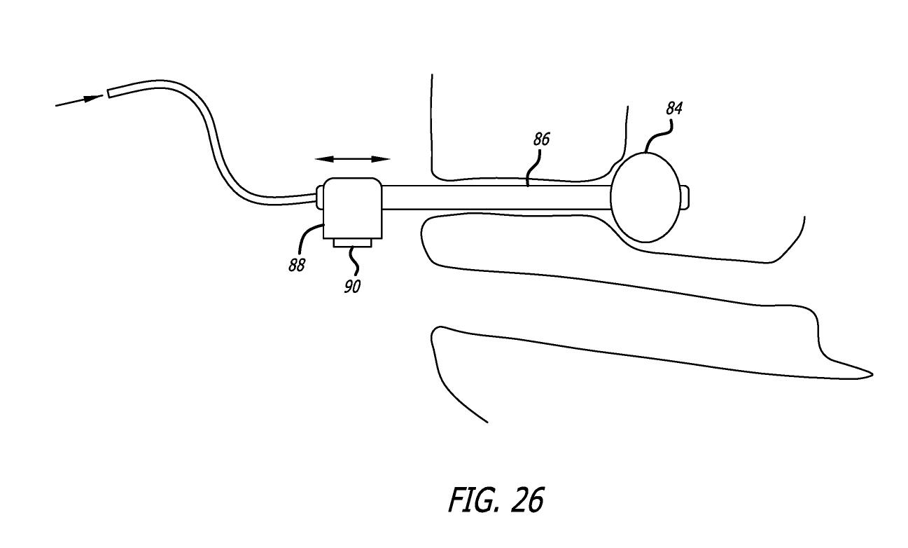

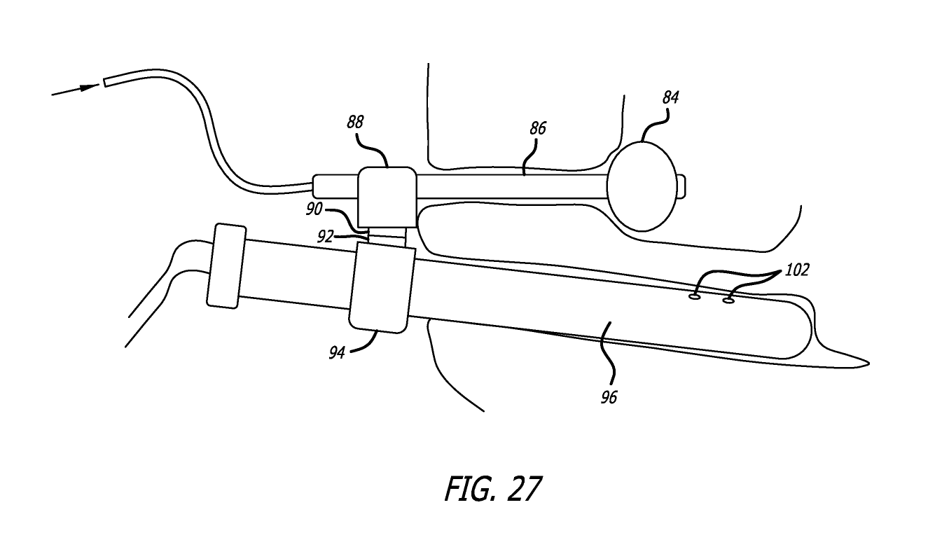

In use, the inflatable balloon 84 is placed in the bladder, inflated, and seated by the bladder neck, as shown in FIG. 26. The urethral guide/handle 88 may then be placed to the urethral opening and secured. The vaginal guide/handle 94 can then be secured at a position along the body of the treatment device 96 to predetermine the position that the energy delivery element(s) will be located within the vaginal wall. The treatment device 96 is then positioned in the vagina, and the two guide/handle portions 88 and 94 can be mechanically secured together using mating portions 90 and 92, for example by magnets or mechanical engagement as seen in FIG. 27. Energy delivery element(s) 102 are now precisely positioned relative to the bladder neck.

Many of the above embodiments are particularly suitable for vaginally delivered treatment of the trigone-associated nerves as they course more laterally from the trigone. However, it is also contemplated that vaginally delivered treatments are also suitable for treatment of the trigone-associated nerves as they course from the trigone region to the tissues deeper of the trigone and within the bladder wall, or in the adventitial tissues between the bladder and vagina, below the trigone region. Below are several embodiments suitable for such treatments.

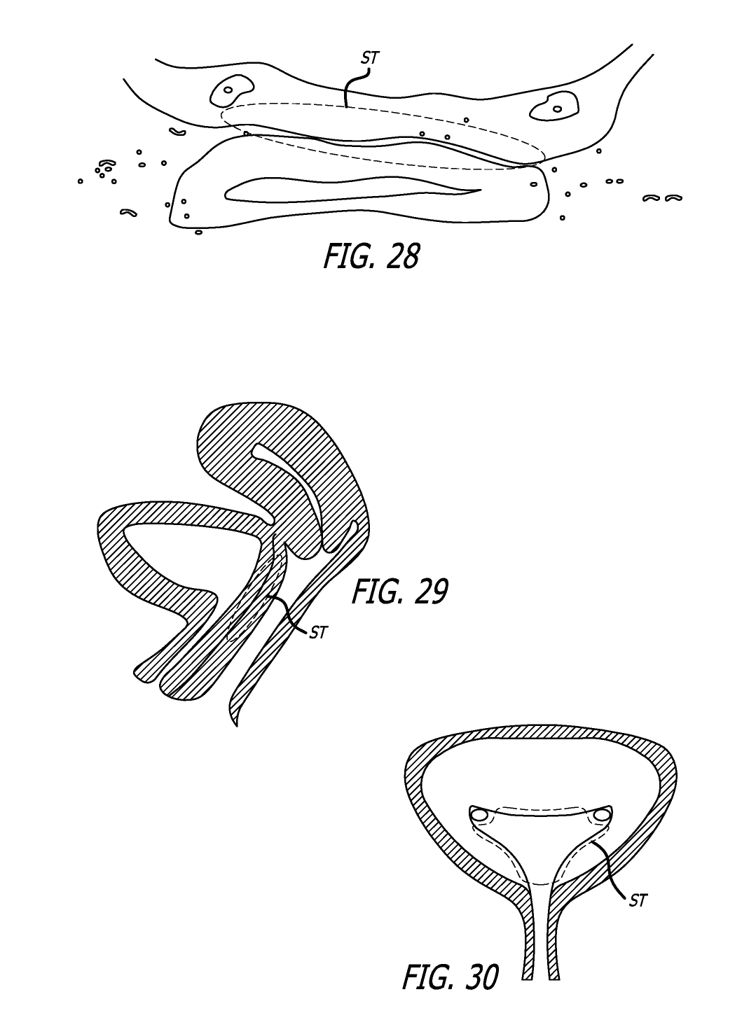

FIG. 28 illustrates a histological cross-section of the bladder and vagina, similar to FIG. 3. The dotted line roughly shows a “sub-trigone” region of interest ST for treatment of trigone-associated nerves as they course from near the surface of the bladder trigone to deeper tissues within the wall of the bladder, to the adventitial layers between the bladder and vagina. Treatment of nerves in or near this region of interest may favorably impact bladder function, e.g. provide relief from bladder conditions such as overactive bladder.

FIGS. 29 and 30 show this “subtrigone” region of interest in side view and axial view, respectively. Note that this region does not have sharp distinct boundaries. Furthermore treatments that target nerves in this region of interest need not treat the entire region of interest, nor must they treat tissue only within this region of interest.

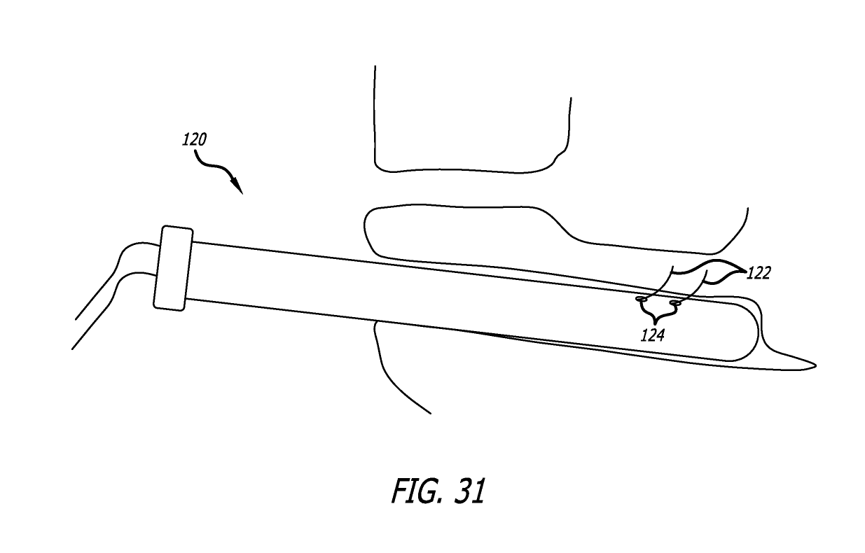

One embodiment 120 that may treat nerves within the treatment zone is illustrated in FIG. 31. This embodiment is similar to that described in connection with FIG. 7, but has one or more energy delivery elements 122 that penetrate toward and into the above described “subtrigone” region of interest. Energy delivery elements may be configured to deliver radio frequency (RF) energy, resulting in localized heating of tissue. Energy delivery element(s) may be configured to be advanceable through apertures 124 in the device to an extended state (shown) from a retracted state (not shown). After energy delivery, energy delivery element(s) may be retracted into treatment device, and removed from the vagina. Energy delivery elements may be electrodes configured to deliver monopolar or bipolar RF energy.

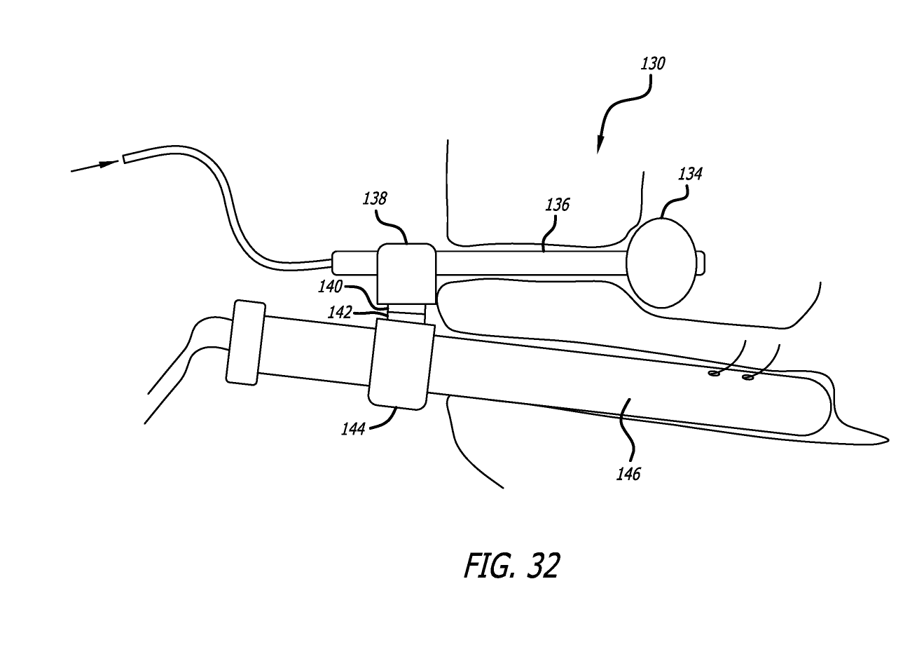

FIG. 32 illustrates an embodiment 130 similar the embodiment of FIG. 31 above, but including a location aid relative to the bladder, similar to the embodiment of FIGS. 25, 26 and 27. Inflatable positioning balloon 134 is on a shaft 136 configured for trans-urethral placement at the bladder neck. A urethral guide/handle 138 is adjustably securable to the shaft 136, and includes a mating portion 140, to be mated with a corresponding mating portion 142 of a vaginal guide/handle 94 that is adjustably securable to a vaginal treatment device 146. Such a location aid could also be used with other embodiments of an energy delivery device described elsewhere herein.

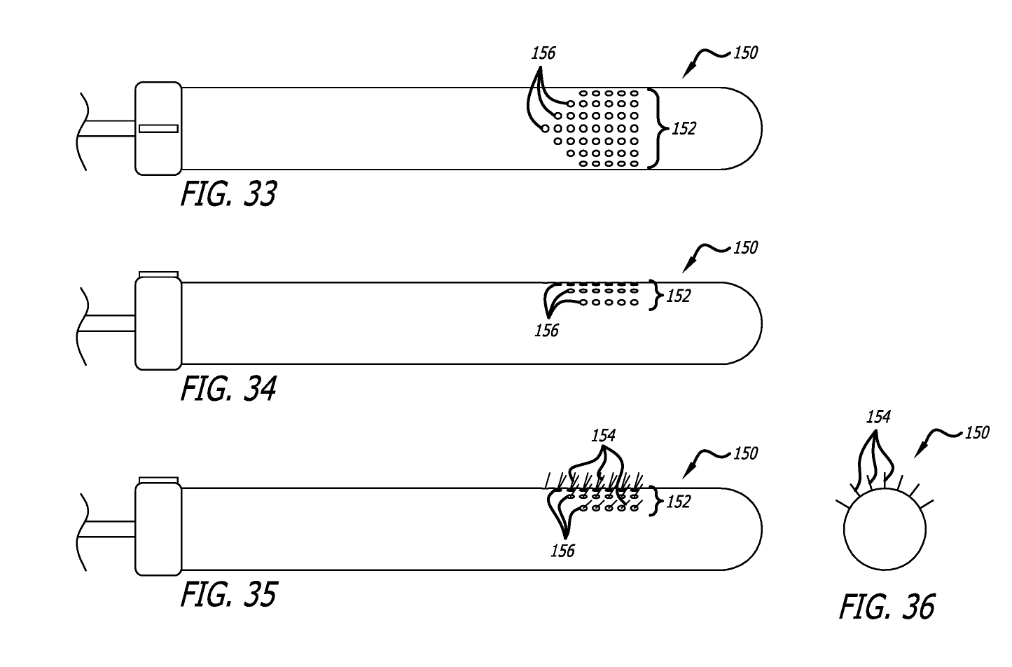

FIGS. 33 through 36 show another embodiment of an energy delivery device 150 configured for treatment of tissue in a subtrigone region of interest. Energy deliver device includes an array 152 of energy delivery elements 154, such as RF electrodes, which can extend from the body of the device. The energy delivery elements are arranged in a pattern, such as the generally triangular shape of the bladder trigone. Once in position within the vagina, the energy delivery elements may be extended (FIGS. 35 and 36) through apertures 156 to penetrate the tissue of the vaginal wall to a depth in or near the region of interest. Energy is applied to the energy delivery elements to treat the tissue, then they may be retracted (FIGS. 33 and 34) to facilitate device removal. The energy delivery elements may be extendable all at the same time and connected to a single control rod (as shown), or they can be extended singularly or in particular groups, with appropriate control mechanisms associated with the energy delivery elements. As suggested above, a location aid such as described in FIG. 32 could also be utilized.

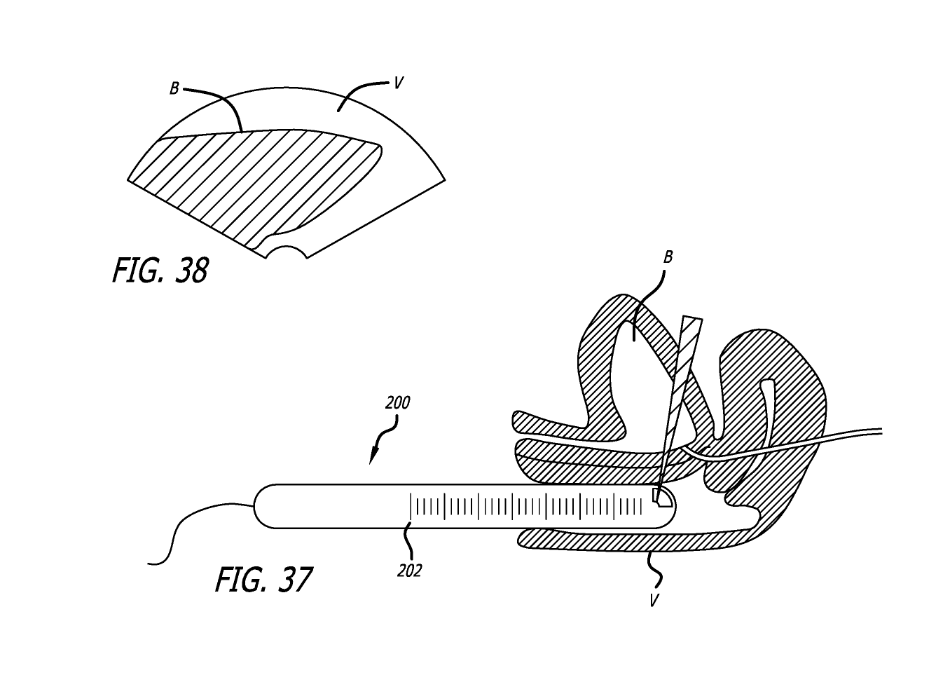

It may be advantageous to position the trans vaginal treatment devices described in this specification to a particular location relative to the bladder ureteric orifices, as for certain embodiments the avoidance of excessive thermal damage to the ureters may be desired. Positioning of treatment devices using ultrasound imaging (or other imaging which can identify the depth and position of the ureters and ureteric ostia) may be employed. FIGS. 37, 38, 39 and 40 illustrate the use of trans-vaginal ultrasound to facilitate such positioning relative the ureters.

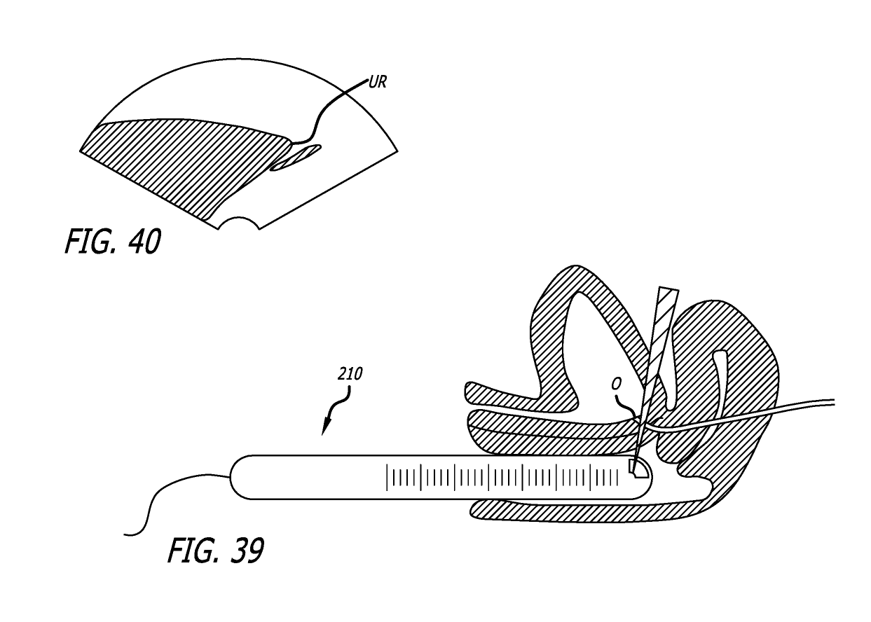

In FIG. 37, an ultrasound imaging wand 200 with corresponding depth indicators 202 is inserted into the vagina V. An image showing the axial cross section of the vagina V is shown in FIG. 38. Note that in this position, the bladder interior B is identifiable. As the wand is inserted deeper, in FIG. 39, the image (FIG. 40) shows one of the ureters UR as well, so the depth and position can be noted. The process may be repeated to identify the other ureter and ureteteric ostium. Such information can be used to accurately position subsequently the various embodiments of transvaginal energy delivery devices described herein.

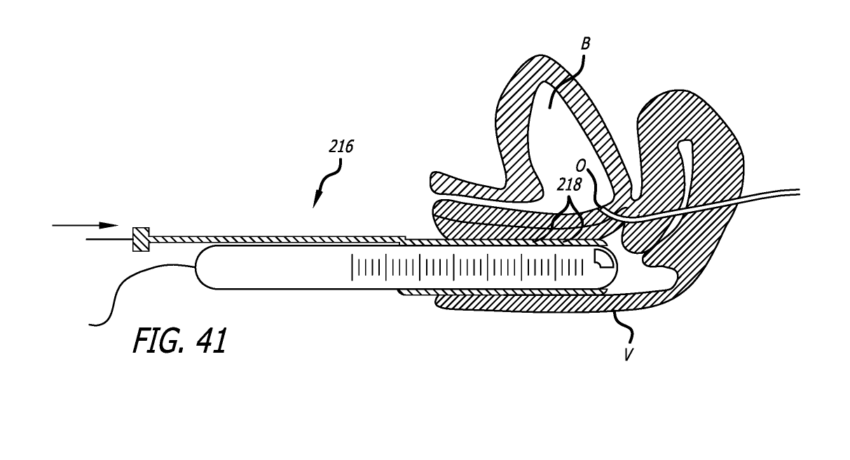

Another embodiment of energy delivery device 210 utilizing ultrasonic imaging is shown in FIG. 41. An ultrasound wand 210 is positioned relative the urereteric ostia O as described above. An energy delivery device 216 having a body that surrounds the ultrasound probe 210 is then advanced to a predetermined position along the ultrasound wand. One or more energy delivery elements 218 may then be extended into the region of interest to treat the tissue.

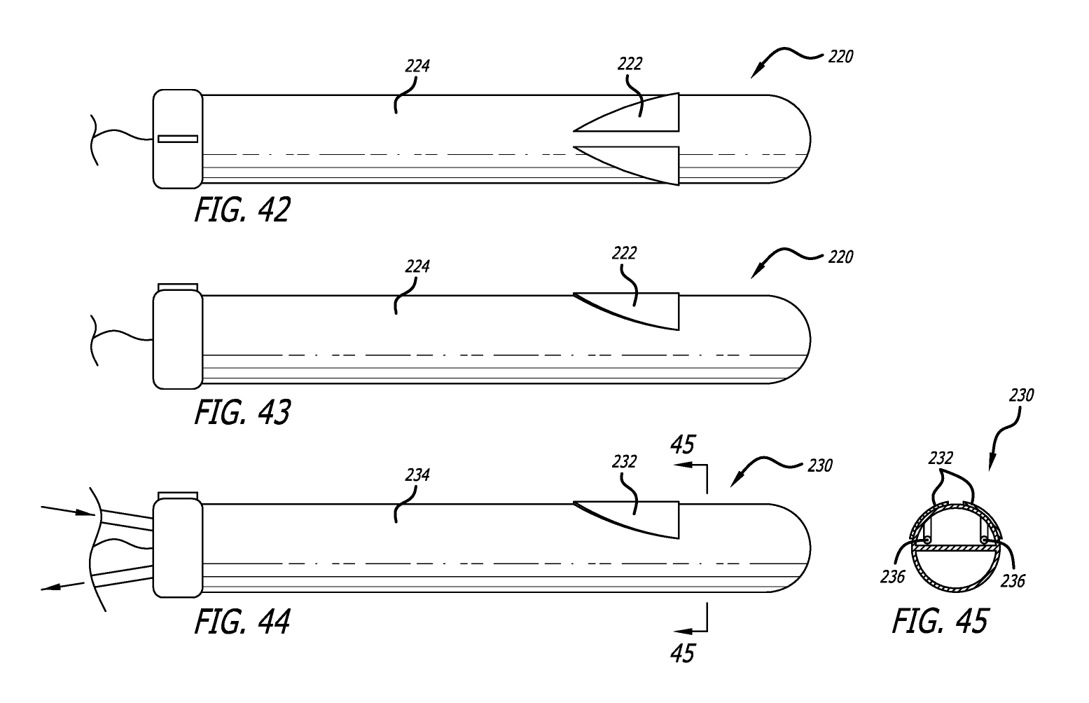

FIGS. 42 and 43 illustrate another embodiment 220 of treatment device. One or more surface electrodes 222 are positioned on the body 224 of the device. They may be shaped and sized to treat the subtrigone region of interest. The electrodes may be configured to delivery RF energy, in either a monopolar (if a single or multiple surface electrode) or bipolar mode (if multiple electrodes). The treatment device may be positioned with the aid of a positioning device or imaging device as described elsewhere.

Another trans-vaginal treatment device embodiment 230, shown in FIGS. 44 and 45, incorporates one or more surface electrodes 232. Additionally, the body 234 of the device may include internal lumens 236 to facilitate passage of a chilled fluid. Once the treatment device is positioned as desired (with or without positioning aids or imaging as described elsewhere), the surface of the body may be chilled by introducing fluid into and out of the device. Then, the electrode(s) may be activated to treat the region of interest, while leaving the tissues at or near the surface of the vagina relatively untreated.

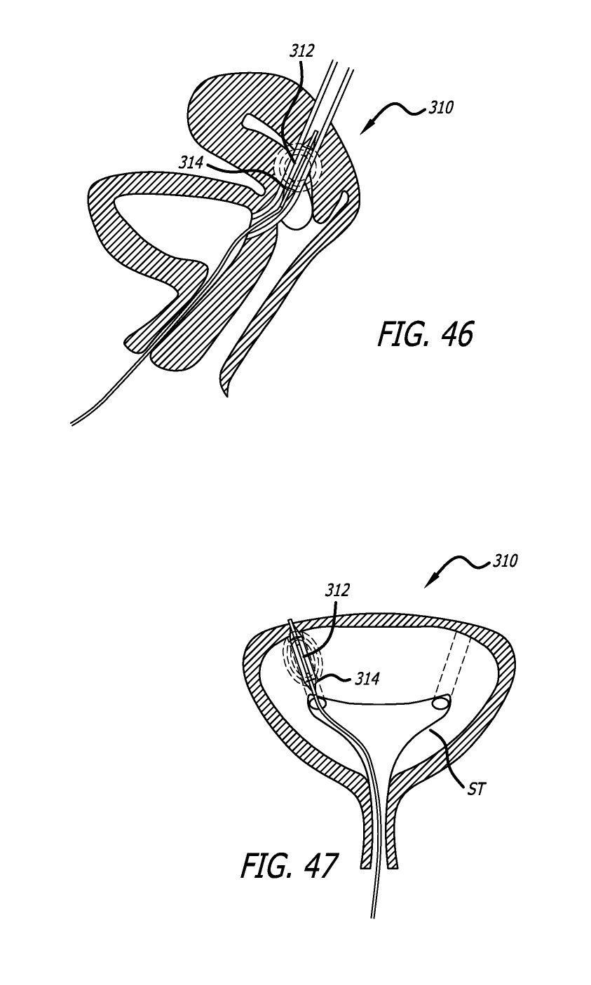

As mentioned above, nerves associated with portions of the bladder, such as the trigone, also coalesce and continue alongside the ureters. Treatment of nerves in this region may also be beneficial. A trans-ureteral treatment device 310 is described in FIGS. 46 and 47. Treatment device 310 may include an inflatable balloon 314 and an energy delivery element 312. Energy delivery element 312 may be configured for delivery of numerous forms of energy, such as microwave energy, ultrasound energy, or RF energy. In the case of microwave energy, a microwave emitter may be disposed within the balloon to radiate thermal energy to the nerve carrying tissues surrounding the ureters. Optionally the balloon 314 may be cooled with cooling fluid to maintain the surface of the ureters, such as the epithelium, cool and limit thermal damage to the epithelial or luminal surface. One or both ureters may be treated, either in one surgical procedure or multiple surgical treatments.

Although the invention has been described in terms of particular embodiments and applications, one of ordinary skill in the art, in light of this teaching, can generate additional embodiments and modifications without departing from the spirit of or exceeding the scope of the claimed invention. For example, it is contemplated that the embodiments could incorporate any suitable form of energy delivery, and/or any suitable form of pharmaceutical agent. Also, combinations of embodiments and treatments are contemplated. Also, combinations of treatment(s) described here with trans-urethral treatment devices described in other of Applicant's patent applications are also contemplated. Accordingly, it is to be understood that the drawings and descriptions herein are proffered by way of example to facilitate comprehension of the invention and should not be construed to limit the scope thereof.

What is claimed is:

1. A method of treating a bladder condition comprising:
placing a treatment device in a patient's vagina such that a front face of the device faces the patient's bladder;
delivering a treatment modality from regions of the device that are lateral to the front face of the treatment device such that said treatment modality affects biological functionality of nerve tissue lateral of the vagina.

2. The method of claim 1 wherein delivering a treatment modality from the treatment device comprises:
placing at least one energy delivery element from the treatment device into a lateral wall of the vagina; and,
delivering energy to tissue within a desired proximity of the delivery element.

3. The method of claim 1 wherein delivering a treatment modality from the treatment device comprises delivering a pharmaceutical agent to a lateral wall of the vagina.

4. The method of claim 2 wherein delivering energy to the lateral wall therefrom comprises delivering RF energy to the lateral wall from the delivery element.

5. The method of claim 2 wherein delivering energy to the lateral wall therefrom comprises delivering microwave energy to the lateral wall from the delivery element.

6. The method of claim 2 wherein delivering energy to the lateral wall therefrom comprises delivering ultrasonic energy to the lateral wall from the delivery element.

7. The method of claim 1 further comprising delivering said treatment modality from said front face of the treatment device.

8. A method of treating a bladder condition comprising:
placing a treatment device in a ureter;
treating nerve tissue in a region away from the epithelium of the ureter while protecting the epithelium from treatment.

9. The method of claim 8 wherein treating nerve tissue in a region outside of the ureter comprises:
placing at least one energy delivery element in a lateral wall of the ureter; and,
delivering energy to the lateral wall from the delivery element.

10. The method of claim 8 wherein treating nerve tissue in a region outside of the ureter comprises delivering a pharmaceutical agent to a lateral wall of the ureter.

11. The method of claim 9 wherein delivering energy to the lateral wall therefrom comprises delivering RF energy to the lateral wall from the delivery element.

12. The method of claim 9 wherein delivering energy to the lateral wall therefrom comprises delivering microwave energy to the lateral wall from the delivery element.

13. The method of claim 9 wherein delivering energy to the lateral wall therefrom comprises delivering ultrasonic energy to the lateral wall from the delivery element.

14. The method of claim 8 wherein placing a treatment device in a ureter comprises placing an expandable balloon in a ureter, said balloon including at least on energy delivery element.

15. The method of claim 8 further comprising cooling a surface of said ureter.

16. A method of treating overactive bladder (OAB) in a female patient comprising:
placing a treatment device having at least one surface electrode in the vagina to a position where the at least one electrode is faces the trigone region of the bladder, the device having an internal lumen configured for receiving a cooling fluid;
activating the at least one electrode to affect nerve functionality;
infusing cooling fluid into the internal lumen.

17. The method of claim 16 wherein the step of activating the at least one electrode occurs prior to the step of infusing cooling fluid into the internal lumen.

18. The method of claim 16 wherein the step of activating the at least one electrode occurs after the step of infusing cooling fluid into the internal lumen.

19. The method of claim 16 wherein the step of activating the at least one electrode occurs simultaneously with the step of infusing cooling fluid into the internal lumen.

20. The method of claim 16 wherein activating the at least one electrode comprises activating at least two surface electrodes that operate in a bipolar RF mode

21. A method of treating overactive bladder (OAB) in a female patient comprising:
placing a visualization device in the vagina to a position adjacent the bladder;
Identifying the location of a ureter and noting the position of the ureter relative to the patient's anatomy or the position of the visualization device;
deploying at least one energy delivery element to a position relative to the ureter,
activating the energy delivery element to treat nerve tissue associated with OAB.

22. The method of claim 21, wherein the energy delivery element comprises an RF electrode and the electrode is deployed into the vaginal wall to a position adjacent the trigone region of the bladder.

23. The method of claim 21, wherein the energy delivery element comprises a surface RF electrode and is positioned against the vaginal wall to a position adjacent the trigone region of the bladder.
